(19) United States
(12) Patent Application Publication
(10) Pub. No.: US 2009/0143863 A1
(43) Pub. Date: Jun. 4, 2009

(54) METHOD FOR DISC REGENERATION USING STEM CELL DERIVED CHONDROPROGENITORS

(75) Inventor: Miguelangelo J. Perez-Cruet, Bloomfield, MI (US)

Correspondence Address: MILLER IP GROUP, PLC;MI4 SPINE, LLC, 42690 WOODWARD AVE., SUITE 200, BLOOMFIELD HILLS, MI, 48304, US

(73) Assignee: MI4SPINE, LLC, Bloomfield Village, MI (US)

(21) Appl. No.: 12/368,021

(22) Filed: Feb. 9, 2009

Related U.S. Application Data
(63) Continuation-in-part of application No. 11/756,274, filed on May 31, 2007.
(60) Provisional application No. 60/846,437, filed on Sep. 22, 2006.

(51) Int. Cl. A61F 2/44; A61B 19/00; C12N 5/02

(52) U.S. Cl. 623/17.16; 128/898; 435/378

(57) ABSTRACT

A method for restoring a degenerated intervertebral disc. The method includes preparing a material including embryonic stem cells, placing the material in the degenerated disc and causing the material to generate notochordal cells in the disc to regenerate the disc. Preparing the material includes differentiating the embryonic stem cells into chondroprogenitors.

### CROSS-REFERENCE TO RELATED APPLICATIONS

This application is a continuation-in-part application of U.S. patent application Ser. No. 11/756,274, filed May 31, 2007, titled “Method for Providing an InVivo Model of Disc Degeneration, claiming priority to U.S. Provisional Application Ser. No. 60/846,437, filed Sep. 22, 2006, entitled “Method for Providing an InVivo Model of Disc Degeneration.”

### BACKGROUND OF THE INVENTION

1. Field of the Invention

This invention relates generally to a method for providing disc regeneration and, more particularly, to a method for providing disc regeneration using embryonic stem cell derived chondroprogenitors.

2. Discussion of the Related Art

The treatment of degenerative disc disease and associated spine ailments is one of the most costly medical conditions with an estimated annual direct cost in the United States of 33 billion and total annual societal cost exceeding 100 billion dollars. Indeed, in one's lifetime most individuals will experience an episode of significant back and or neck pain. Although most individuals will improve with non-operative intervention, a significant percentage will go on to require costly surgery or other medical intervention.

The etiology of chronic back pain is multi-factorial, but in a significant proportion of patients, degenerative disc disease is the underlying cause. As an individual ages, the intervertebral disc looses water, begins to collapse, and can ultimately fail to adequately support adjacent vertebrae. As a result, the neural element can become compressed within the neural foramen as well as the central canal of the spine leading to painful back conditions. Additionally, discogenic back pain, perhaps a less well understood condition, can also lead to painful back conditions as a result of disc degeneration.

The process of intervertebral disc degeneration occurs in all of us as we age and its treatment in symptomatic patients has significant socioeconomic impact. Many studies have shown that notochordal cells, the precursors of the disc, are no longer present after age 10. During embryogenesis, notochordal cells are believed to be responsible for the formation of spine and intervertebral disc, as well as for maintenance and metabolic control of the nucleus pulposus (NP) further in life. The relationship between loss of notochordal cells with age and the onset of disc degeneration can perhaps best be understood by the changes in biomechanics of the discs as a consequence of proteoglycan loss. The proteoglycans are the hydrophilic moiety of the intervertebral disc. These molecules are uniquely structured to hold water and therefore provide the cushioning quality of the intervertebral disc. It has been shown in recent studies that notochordal cells produce 1.5 fold more proteoglycans and extracellular matrix than terminally differentiated chondrocytes. As the notochordal cells differentiate to chondrocytes in the NP, less water holding proteoglycan matrix is available. A cascade of events ensues resulting in disc degeneration, desiccation and collapse. Consequently, the annulus fibrosis (AF) begins to fissure and cracks, contributing to a vicious cycle of disc degeneration potentially resulting in chronic lower back pain.

The intervertebral disc is comprised of an external AF made up primarily of lamellar bands of type I collagen that surrounds a soft gelatinous central NP made up primarily of type II collagen and a proteoglycan matrix. The proteoglycan moiety is a highly hydrophilic molecule capable of holding significant amounts of water. The water holding capacity of the NP, held within the confines of the intact AF, gives the intervertebral disc its unique function, particularly that of providing a mobile compressible distraction between adjacent vertebral bodies, and thus providing the unique flexibility of the spinal column.

There are primarily two cell types associated with the NP, namely, the notochordal cell and the chondrocyte. During embryogenesis the intervertebral disc develops from the embryonic mesenchyme and notochord. During this process the notochordal cells becomes discontinuous within the outer AF of the disc and are felt to lead to the creation of the NP. Histologically notochordal cells appear as large cells with granular cytoplasmic inclusions giving them the name physolipherous cells or “bubble cells.” However, the process by which the notochordal cell forms the NP is largely unknown. It is felt that these cells also produce the proteoglycan matrix which holds the water molecules that is so important in maintaining the viable function of the intervertebral disc, i.e., supporting adjacent vertebrae. Human as well as animal studies have shown that with age, the notochordal cell population disappears. It is not certain, but notochordal cells may terminally differentiate into chondrocytes. Thus, the notochordal cell may represent a stem cell population within the NP much like the mesenchymal cell seen within the bone marrow that might terminally differentiate into chondrocytes seen within the NP in older animals. Since the notochordal cell is felt to produce the proteoglycan water holding matrix of the intervertebral disc, terminal differentiation of these cells could initiate the process of disc degeneration. The cells of the mature NP in adult humans as well as many species of mature aged animals are primarily small terminally differentiated chondrocytes.

### SUMMARY OF THE INVENTION

In accordance with the teachings of the present invention, a method for restoring a degenerated intervertebral disc is disclosed. The method includes preparing a material including embryonic stem cells, placing the material in the degenerated disc and causing the material to generate notochordal cells in the disc to regenerate the disc. Preparing the material includes differentiating the embryonic stem cells into chondroprogenitors.

Additional features of the present invention will become apparent from the following description and appended claims, taken in conjunction with the accompanying drawings.

### BRIEF DESCRIPTION OF THE DRAWINGS

FIG. 1 is a perspective view of a needle being percutaneously inserted into a disc of an anesthetized rabbit;

FIG. 2 is a close-up view of the needle inserted into the disc of the rabbit;

FIG. 3 is a fluoroscopic image of a needle being positioned into the disc of a rabbit;

FIG. 4 is a plan view of the rabbit being imaged by an MRI device;

FIG. 5 is an MRI image of a rabbit showing InVivo created disc degeneration in a rabbit spine model and normal appearing rabbit intervertebral discs;

FIG. 6 is an MRI of a degenerated disc at L₅S₁ in a human spine;

FIG. 7 is a photomicrograph at low-power showing notochordal-type cells seen in degenerated intervertebral discs implanted with ESC derivatives;

FIG. 8 is a photomicrograph at high-power showing notochordal-type cells seen in degenerated intervertebral discs implanted with ECS derivatives;

FIG. 9 is a photomicrograph at low-power showing ossifying chondrocytes in a degenerated disc;

FIG. 10 is a photomicrograph at high-power showing ossifying chondrocytes in a degenerated disc;

FIG. 11 is a photomicrograph showing PAS-positive cords and groups of notochordal-type cells;

FIG. 12 is a photomicrograph showing reduced PAS activity following glycogenase exposure of cords and groups of notochordal-type cells;

FIG. 13 is a photomicrograph showing pankeratin-positive activity of notochordal-type cells in a disc implanted with ECS derivatives; and

FIG. 14 is a photomicrograph showing a confocal fluorescent view of a disc having a green fluorescent pattern of ECS-derived notochordal-type cells.

### DETAILED DESCRIPTION OF THE EMBODIMENTS

The following discussion of the embodiments of the invention directed to a method for disc regeneration using stem cell derived chondroprogenitors is merely exemplary in nature, and is in no way intended to limit the invention or its applications or uses.

From the discussion above, there is an interest in the differentiation of embryonic stem cells (ESCs) into notochordal cells within the environment of a degenerated intervertebral disc as a method of disc regeneration. In order to study the differentiation of ESCs into notochordol cells, a disc degeneration model is necessary. A number of animal models of disc degeneration exist, however, the rabbit model is chosen with modifications in technique to preserve the NP. The discussion below proposes a process for providing an InVivo model of disc degeneration, where the disc degeneration occurs relatively rapidly in the model, and can be easily studied, to learn the processes of human disc degeneration, which typically occurs slowly.

Because ESCs have the ability to differentiate along different cell lines, they represent a possible source of notochordal cells. When a stem cell divides it can embark along a course of a particular cell line or remain a stem cell. The use of ESCs as a possible therapy for disc regeneration is a recent development. Investigators have studied the use of mesenchymal stem cells for intervertebral disc regeneration. However, stem cells have not been used to investigate intervertebral disc regeneration in an In Vivo model of NP degeneration. Researchers have shown mesenchymal stem cells could be induced to development towards a NP-like phenotype by creating a growth environment, i.e. hypoxia, similar to that found in the intervertebral disc. Further, ESCs were differentiated towards the chondrogenic lineage in the presence of selective media culture with TGF-β, dexamethasone, and ascorbate. It has been hypothesized that by placing pre-treated ESCs in a degenerated disc environment they would be encouraged to differentiate along a new disc material lineage. Since it is felt that the notochordal cell is the first cell line seen during the embryogenesis of the NP, it was also hypothesized that the notochordal cell type would indeed be the first cell line seen histologically, as new disc material is formed. Later in the developmental process of the disc, these notochordal cells might terminally differentiate into chondrocytes. Therefore, the discussion herein includes an investigation of implantation of ESCs into a degenerated intervertebral disc and production of cells similar to those seen in the embryogenesis of the disc, namely, notochordal cells.

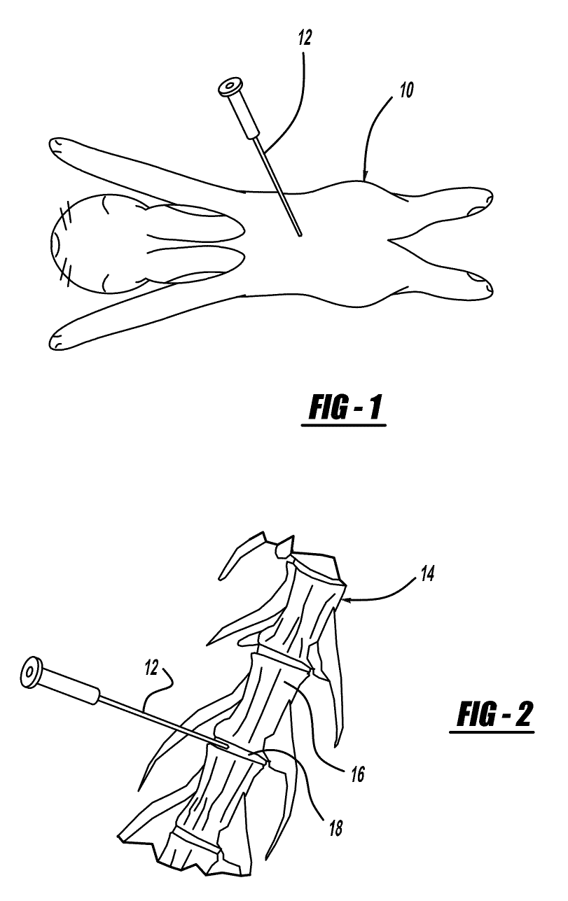

FIG. 1 is a perspective view of a rabbit 10 that has been anesthetized, where one or more of the intervertebral discs in the rabbit 10 will be the InVivo model. A technician will percutaneously insert, i.e., through the skin, a needle 12 into the rabbit 10 towards an intervertebral disc of the rabbit 10. FIG. 2 is a perspective view of a spine 14 of the rabbit 10 including vertebrae 16 and discs 18 therebetween. The needle 12 is shown inserted into one of the discs 18 of the rabbit's vertebrae 16, where the needle 12 ruptures the annulus of the disc 18. By rupturing the disc in this fashion, the inner gelatinous portion of the disc begins to dry out and desiccate causing the disc to degenerate. In this non-limiting embodiment, the needle 12 is a 16-gage needle because it is a proper size for the height of the disc 18. However, in other embodiments, other needle sizes may be more applicable.

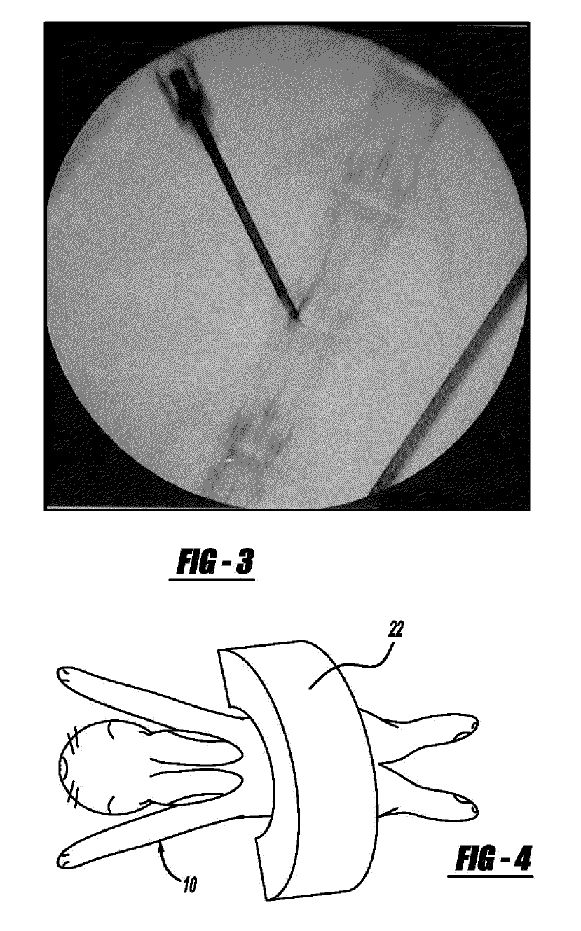

The technician can use fluoroscopic X-ray images to allow the technician to visualize the location of the needle 12 in the rabbit 10 so that the technician is able to properly position the needle 12, as shown. FIG. 3 is a fluoroscopic X-ray image showing a needle positioned within the disc of a rabbit for the purposes described herein. Other methods for guiding the needle 12 can also be employed, such as X-rays, computer tomography, tomograms, MRIs, etc.

By puncturing the annulus of the disc in an animal model, disc degeneration will begin to occur. This procedure may also be used to induce disc degeneration in other animal models, including primates. For humans, disc degeneration from old age or disc damage occurs relatively slowly over several years. However, with a suitable animal model, disc degeneration occurs much more rapidly, typically on the order of a few weeks. Although other lab animals can be used as the model, such as rats, mice, sheep, pigs, primates, etc., rabbits are used as the more preferable lab specimen because they are relatively easy to work with and are of a large enough size. Further, because a rabbit has a relatively upright posture when sitting, it mimics the loading of the lower spine intervertebral disc that is similar to a human.

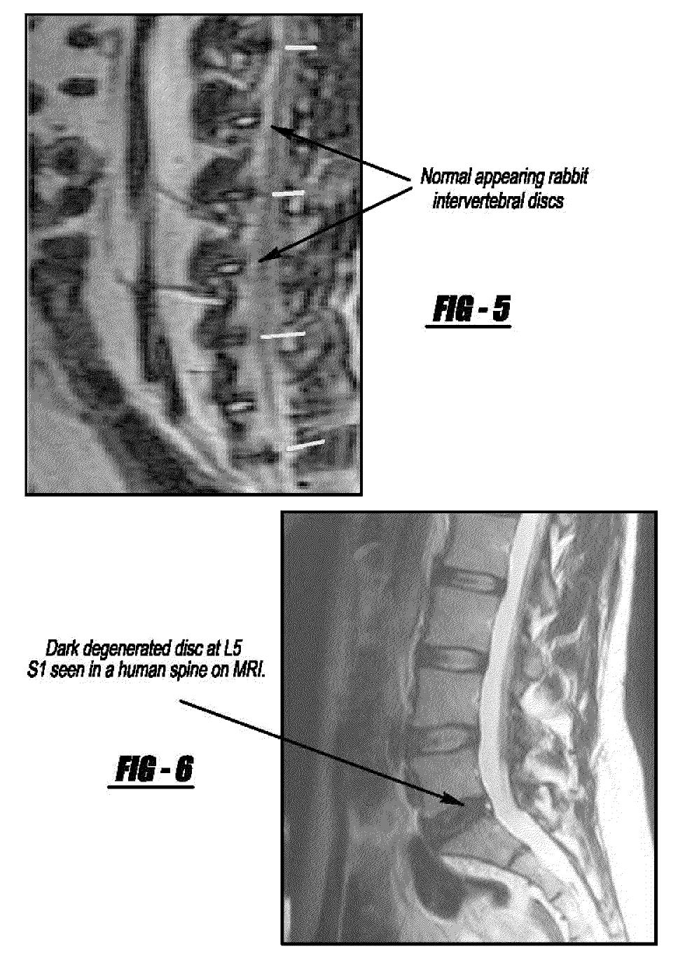

Once the disc 18 has been ruptured by the needle 12, and the degeneration process begins, its progress can be followed by magnetic resonant imaging (MRI) of the rabbit 10. FIG. 4 is a plan view of an MRI device 22 taking images of the rabbit 10 for this purpose. Other imaging device may also provide suitable images, such as image guidance technologies, computer tomography, tomograms, etc. FIG. 5 is an MRI of an animal rabbit model that has degenerated discs as a result of the process discussed above. FIG. 6 is an MRI of a human spine showing a degenerated disc at L₅S₁ in that it is similar to the degenerated disc in the rabbit model.

Once the disc degeneration model has been produced, then various therapeutic drug experimentations and procedures can be used to treat the degenerated disc, which ultimately may be used on humans having degenerated discs. Thus, the efficacy of various disc regeneration technologies can be tested using this model to determine their efficacy and safety when applied clinically. In one such treatment/scenario, notochordal cells and chondrocytes can produce cartilage for disc regeneration. These cells can ultimately develop into a new disc. Additionally, various therapies can be implemented to treat the disc degeneration in the rabbit 10. For example, various drugs can be developed that can be experimentally used on the rabbit 10 to determine whether they have an effect on reducing the speed of the disc degeneration and/or reversing the disc degeneration. Further, various therapies can be used to try to rehydrate the disc by injecting water holding drugs and other materials into the disc. Also, the rabbit disc degeneration model can be used for various other analyses and studies concerning disc degeneration including novel devices and instrumentation.

The following is a more detailed discussion of the process of the disc study discussed above. In this study, 16 skeletally mature female New Zealand white rabbits, at age 6-12 months, weighing 3.2-3.5 kg, were used. An initial magnetic resonance imaging (MRI) was performed on all rabbits under sedation to confirm that no previous disc degeneration was present either in the control or experimental discs.

Each rabbit was initially tranquilized by intramuscular injection of xylazine (3 mg/kg) and ketamine (40 mg/kg), and then placed on supplemental oxygen. The rabbits were shaved from the sacroiliac portion of the mid-back of the animal. Under general anesthesia, using inhalation of 2% isofluorane, the field was prepped with betadine, and draped in surgical fashion. A mini-fluoroscopic unit was then used to identify the levels in the lumbar spine. A lateral approach from the rabbit's right flank was taken to enter the disc segment of interest. The overlying skin was first anesthetized with 0.75% bupivacaine and a 16 gauge needle was then advanced into the disc space through Kambin's triangle starting approximately 3 cm off the midline at the level of interest. Antero-posterior (AP) and lateral fluoroscopic imaging were used to guide needle placement. The needle was advanced until fluoroscopic confirmation showed the needle tip to be in the center of the disc. Needle punctures of two adjacent discs at L2, 3 (Group B) and L3, 4 (Group C) experimental groups were performed to induce disc degeneration. The L4, 5 and L5, 6 (Group A) disc levels served as control. Following the procedure, the rabbits where returned to their respective cages (4000 cm²), with food and water provided on demand for eight weeks.

The rabbits were tranquilized with an intramuscular injection of ketamine (40 mg/kg) and xylazine (3 mg/kg), placed supine within the MRI coil (General Electric Medical Systems). A localizing mid-sagittal T-2 weighted image (T_{R}, 2500 milliseconds; T_{E}, 100 milliseconds) was taken to view L1-2 through L5-6 intervertebral levels. Next, 3-mm thick mid-sagittal sections were taken using T-2 weighted imaging sequences (T_{R}, 2500 milliseconds; T_{E}, 100 milliseconds) to evaluate signal characteristics within the intervertebral disc. T-2 weighted imaging sequences (T_{R}, 5200 milliseconds; T_{E}, 100) were taken through each lumbar intervertebral disc. MRI evaluations were performed initially, and then post-operatively at 2, 6, and 8 weeks.

Mouse ESCs (7AC5), originally obtained from ATCC (Manassas, Va.), were maintained using a previously described protocol. These cells were further grown in dulbecco's modified eagle medium (DMEM) (Invitrogen, Carlsbad, Calif.) containing 10% fetal bovine serum (FBS) (Invitrogen, Carlsbad, Calif.), 0.1 mm 2-mercaptoethanol (Sigma-Aldrich, St. Louis, Mo.), and 5.6% of the supplement (gentamicin, 0.1%, streptomycin, 0.2% and penicillin, 0.12%). In order to maintain the cells in an undifferentiated state, the medium was supplemented with 1,000 U/ml of leukemia inhibitory factor (LIF; Chemicon International Inc., Temecula, Calif.), and mouse embryonic fibroblasts (MEF). MEF were developed from embryos of mice (CF strain). The MEF were maintained and grown following standard culturing techniques. The ESCs were cultured on 0.1% gelatin-coated tissue culture plates as previously described. Also, a mutant green fluorescent protein (GFP) was used to identify the ESCs, once they were injected into the intervertebral disc. Fluorescent microscopy (Olympus) and confocal microscopy were used to confirm fluorescent labeling of the cells prior to implantation as well as to identify GFP expressing cells after the implantation into the rabbit NP.

ESCs were incubated on gelatin-coated tissue culture plates in a selective medium, supplemented with a mixture of Tumor Growth Factor-β (TFG-β, 5 ng/ml), ascorbic-acid phosphate (50 μg/ml), and insulin-like growth factor (10 μg/ml) and lacking leukemia inhibitory factor. The medium was changed every two days. In order to direct the transformation of these cells along a chondrocyte lineage the ESCs were treated with cis-retinoic acid (1×10⁻⁷ M). The differentiated ESCs were monitored by light microscopy on a daily basis. After 12-14 days of incubation in the medium, chondroprogenitor cells were detected. At the end of 4 weeks, the differentiated chondrogenic cells were removed and injected into the degenerated intervertebral discs of the Group C rabbits. The experimental Group C disc had previously been punctured to induce disc degeneration confirmed using MRI imaging.

Alcian blue (Sigma-Aldrich, St. Louis, Mo.), a proteoglycan and glycoprotein stain was used to detect and distinguish chondroprogenitor ESCs. Both of these components are also major constituents of the extracellular gelatinous proteoglycan matrix found within the NP of the intervertebral discs. Samples were washed with PBS and fixed with freshly-made 4% paraformaldehyde for 30 minutes. The samples were then incubated with 1% Alcian blue in 3% acetic acid for 1 hour, rinsed in distilled water and observed for blue staining.

The rabbits were tranquilized, placed under general anesthesia, and surgically prepped as previously described. The GFP expressing ESC at concentration of 1×10⁶ cells in 20 μl of DMEM solution were prepared for injection. Guided by AP and lateral fluoroscopy, the cells were, then, injected with a 32 gauge 25 ul Hamilton syringe into the L 3, 4 degenerated disc segments (Group C) which was confirmed by MRI.

At 8 weeks post-implantation, the spines were, subsequently, harvested for intervertebral disc processing. Three separate groups (A, B and C) of intervertebral discs were analyzed.

The intervertebral discs were fixed in 10% neutral buffered formalin for 1 week and decalcified with ethylenediaminetetraacetic (EDTA 0.75M, 7.8 pH). The intervertebral discs were embedded into paraffin and cut into axial sections (5 μm thickness) using a microtome. The sections were stained with hematoxylin and eosin (H&E) and analyzed qualitatively under light microscopy (100× and 400×) (Olympus model). Immunofluorescent analysis of both control and experimental group intervertebral disc was performed using a fluorescent lamp objective at low and high power magnification. Confocal microscopy and independent and blinded review of tissue was performed by a board certified pathologist.

MRI confirmed disc degeneration occurred by two weeks post-operatively in all intervertebral discs punctured. Also, as evidenced by MRI studies, the process of disc degeneration progressed until animals were euthanized and resulted in disc height loss, diminished signal intensity as well as progressive decrease in the NP surface area over the 8-week post-operative period. These results are consistent with observation by others. However, unlike other techniques of disc degeneration, the percutaneous technique maintained the NP within the center of the intervertebral disc and no extruded or herniated disc fragments was observed. Conversely, in the control group, the appearance of the intact discs remained unchanged over the same time period. The height and MRI signal intensity remained unchanged in the control healthy disc. There were no operative complications or deaths associated with this model. The rabbits tolerated the procedure well without any post-operative behavioral problems or neurological signs.

Pre-implantation ESC-derivatives analysis by fluorescent microscopy confirmed that these cells expressed GFP fluorescence prior to implantation. Trypan blue staining confirmed 87% viability of injected cells and alcian blue staining confirmed the production of proteoglycan and glycoprotein confirming these cells where chondroprogenitor cells.

Post-mortem H & E histological analysis of Group A intervertebral disc showed aged chondrocytes and the absence of notochordal cells. The chondrocytes appeared as small cells with dark staining cytoplasm and a shrunken, relatively dense nucleus. Group B discs displayed some fissuring of the annulus fibrosus and generalized disorganization of fibrous tissue within the NP, see FIGS. 9 and 10. Group C discs showed viable new cartilage forming as well as notochordal cell growth.

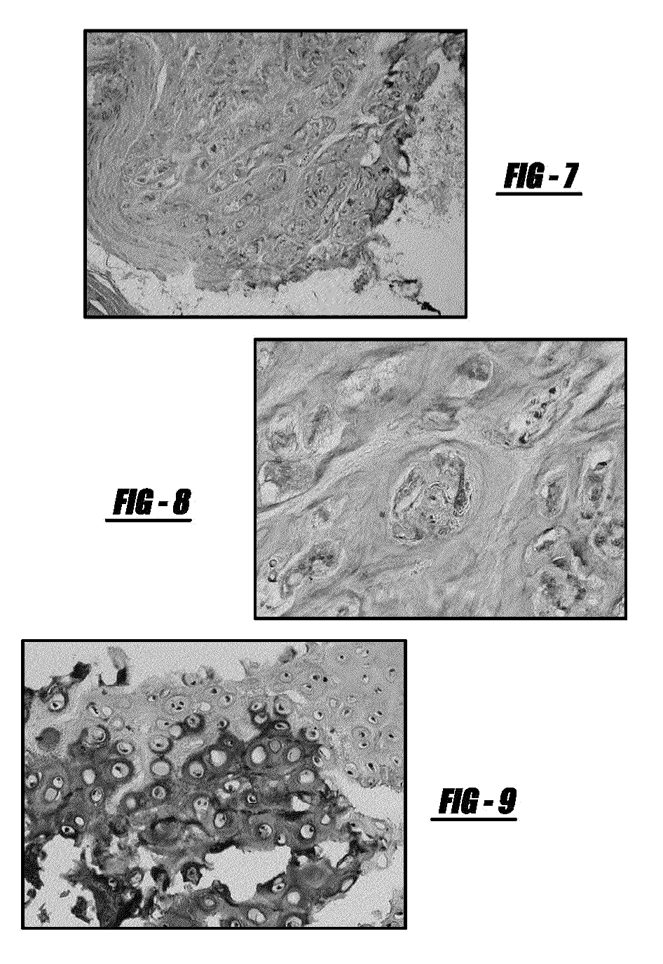

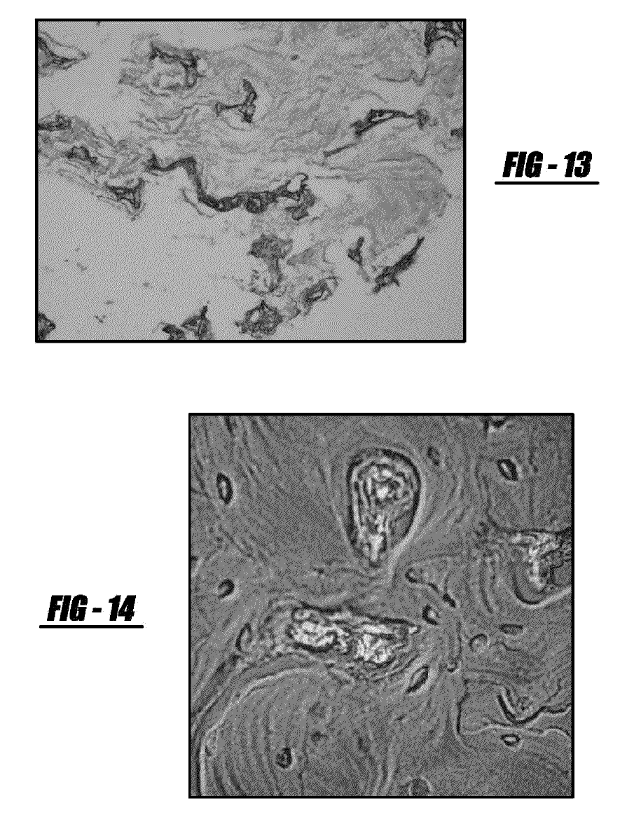

The H & E stained discs of Group C showed that the nucleus pulposus (NP) was focally infiltrated by hyper-cellular groups, principally arranged in cords and occasionally admixed with large lobulated (physaliphorous-type) cells, diffusely separated by pale loose myxoidstroma (H & E). The cell groups are generally composed of 3-6 small round to ovoid nuclei, surrounded by eosinophilic cytoplasm in the corded structures (H&E) and bubbly, foamy cytoplasm in the lobulated cells of physaliphorous-type. Both forms exhibit strong pankeratin activity and did not exhibit significant nuclear pleomorphism or mitoses. There was neither associated necrosis nor inflammatory cells as evidenced by the absence of macrophages or leukocytes. The patterns described strongly resembled that of chordomas, which was consistent with notochordal tissue, see FIGS. 7 and 8. This cell histology was not noted in the control healthy disc (Group A) nor the degenerated disc which was not implanted with ESC-derivatives (Group B). Additionally, cell histology differed from the initial pre-implantation of chondrogenic cells for which the histology has been previously described as spherical fibroblastic morphology. This implied that the chondroprogenitors did differentiate into notochordal tissue after implantation into the degenerated disc. Morphometric analysis to quantify the amount of cells that did differentiate was not done in this study. Confocal fluorescent analysis was negative for Groups A and B but revealed viable fluorescing notochordal cells within experimental Group C discs implanted with ESCs, see FIG. 14. Sixty-micron sections were scanned and the fluorescent pattern of the notochordal tissue was seen through the entire thickness of the cell indicating that these cells originated from the implanted GFP labeled ESC-derivatives. Fluorescence would not have occurred if these cells were not viable and expressing GFP. Of note, no inflammatory response, as evidence of cell mediated immune response, was noted in all three groups. Additionally, there was no evidence of teratoma or “tumor” formation that had been noted in similar chondroprogenitors injected into the mouse subcutaneous tissue as seen in our previous studies.

To induce disc regeneration, ESCs were pre-treated prior to implantation to encourage differentiation along a chondrogenic cell lineage. In previous reports, mouse ESCs were capable of differentiating into chondrocytes via embryoid bodies (EBs) when treated with TFG-β. Therefore, we used a similar technique to treat ESCs before implantation. Additionally, the differentiation of ESCs towards a chondrogenic cell lineage has also been found to be influenced by hypoxic environment in which the cells were cultured. Under hypoxic conditions in vitro, mesenchymal stem cells were found to differentiate along a phenotype consistent with that of the NP. Therefore, we hypothesized that implanting ESC-derivatives into the hypoxic environment of a degenerated disc could potentially encourage differentiation of these cells into viable new disc material.

A number of In Vivo animal models for disc degeneration have been investigated. The model of Lipson and Muir involved the process of creating a full thickness, ventral stab incision in the AF of the rabbit spine using an 11 blade scalpel. However, done in an open surgical fashion, this model had its limitations in that the NP herniated out of the stab incision. To overcome the extrusion of the NP while initiating disc degeneration, Sobajima et al. modified this model by surgically exposing the intervertebral disc via a ventral approach and stabbing the disc with a 16-gauge needle instead of a scalpel blade. Using this model, they were successful in showing a sequential process of disc degeneration that occurred over time and was documented with serial MRI imaging and histologic analysis. In this study, further modification of Sobajima's model was created. A percutaneous disc puncture was performed through Kambin's triangle using anterior posterior and lateral fluoroscopy to guide needle placement. Sequential MRIs of the rabbit spine confirmed reproduction of the degenerative disc process occurred at 2 weeks post-procedure. This model was successful in limiting animal morbidity/mortality, initiating disc degeneration, and preserving the NP, which was the target tissue for regeneration using ESCs. However, species variation makes direct correlation of this model of disc degeneration with the process that occurs in humans difficult. Namely, the rapidity to which degeneration occurs in the rabbit model after annulus fibrosus puncture does not reflect that seen in humans. Nevertheless this model is cost effective, does not appear to harm the animal, and is reproducible making it a much more viable model than using larger animals, i.e., pigs, sheep.

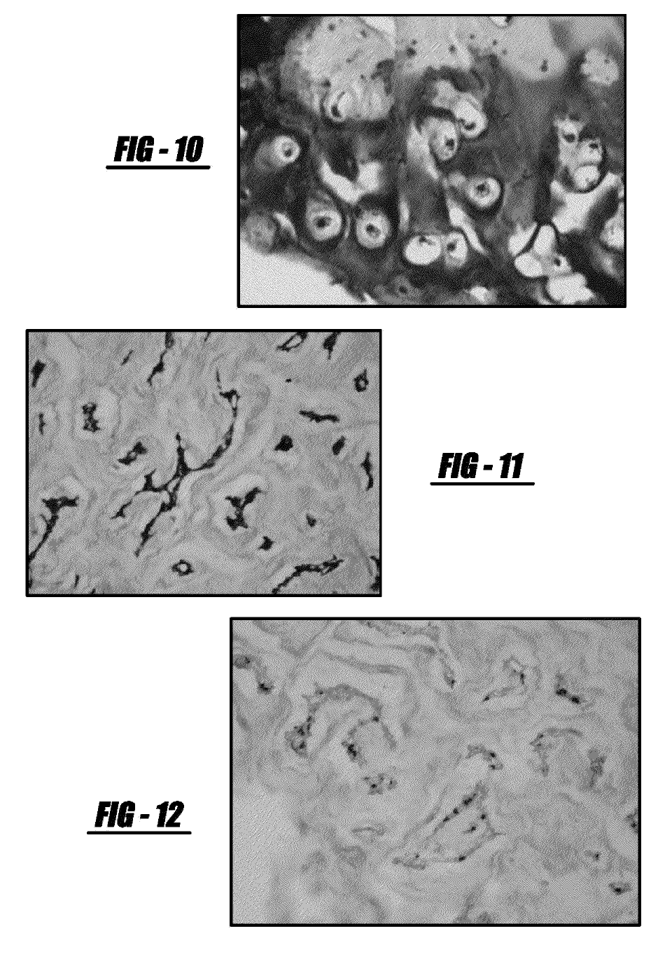

This study also demonstrated that implanted ESC-derived chondroprogenitors could potentially differentiate into notochordal cells. Harvested Group C discs confirmed that eight weeks post implantation, fluorescent labeled cells appeared to resemble notochordal tissue, i.e., cells with large vacuolated cytoplasm, physaliphorous-type cells, see FIGS. 7 and 8. These cell types were not observed in Groups A and B. Additionally, the pre-implanted chondrogenic derivatives of ESCs has a spherical fibroblastic morphology in culture which differed from differentiated ESCs implanted in group C which had a notochordal cell type appearance. Histologically these cells stained as periodic acid-Schiff (PAS) (+) positive cords and had reduced PAS activity following glycogenase exposure (consistent with glycogen) of cords and groups of notochordal-type cells, see FIGS. 11 and 12. They also displayed pankeratin positive activity of notochordal-type cells, see FIG. 13. However, long-term implantation studies are needed to confirm this and are currently underway. Lastly, no endodermal or ectodermal cell lines were seen on histology. Interestingly, all three cells lines were seen in our earlier investigations where ESC-derivatives were placed subcutaneously in a murine model.

Both Groups A and B showed primarily chondrocytes within the NP and no notochordal cell tissue. Additionally, Group B intervertebral disc showed histological changes consistent with a degenerated disc as previously reported, see FIGS. 9 and 10. No inflammatory cells (macrophages, lymphocytes etc) were observed in Group C disc tissue even though the implanted murine ESC-derivatives were xenografts (donor cells from a different animal species). This might be expected since the disc has no blood vessels within it for inflammatory cells to reach the xenograft to cause a cell mediated immune response rejection. Therefore, the intervertebral disc might represent as yet unrecognized immuno-privileged site ideal for xenograft implantation. In the future, this could expand the applications of xenografts for the treatment of disc degeneration similar to porcine valve replacement currently used in humans.

It might be hypothesized that overtime, perhaps months to years, the notochordal cells seen in experimental Group C would continue to differentiate towards terminal chondrocytes. Furthermore, since it is believed that the notochordal cells are largely responsible for the production of the disc proteoglycan matrix, one might suspect a relative increase in proteoglycan content within Group C disc as compared to Groups A and B. The exact function of the notochordal cells in forming the nucleus pulposus and proteoglycans is largely unknown.

Thus, this study illustrates a reproducible model for the study of disc degeneration as well as potential disc regeneration using ESC-derivatives. New notochordal cell populations were seen in ESC injected degenerated discs. The lack of immune response to xeno-transplanted mouse cells in an immune competent rabbit model could points to a previously unrecognized immuno-privileged site within the intervertebral disc. However, the authors recognize that this serves as a preliminary investigation and that we could not provide definite proof as to whether disc regeneration was indeed taking place. Nonetheless, this study does offer interesting insight into the potential for disc regeneration using ESCs and further investigations are warranted.

The foregoing discussion discloses and describes merely exemplary embodiments of the present invention. One skilled in the art will readily recognize from such discussion and from the accompanying drawings and claims that various changes, modifications and variations can be made therein without departing from the spirit and scope of the invention as defined in the following claims.

What is claimed is:

1. A method for restoring a degenerated intervertebral disc, said method comprising:
preparing a material including embryonic stem cells;
placing the material in the degenerated disc; and
causing the material to generate notochordal cells in the disc to regenerate the disc.

2. The method according to claim 1 wherein placing the material into the disc includes injecting the material into the disc using a syringe.

3. The method according to claim 1 wherein placing the material into the disc includes visualizing the material using lateral fluoroscopy.

4. The method according to claim 1 wherein preparing the material includes pretreating the embryonic stem cells to encourage chondrogenic cell lineage.

5. The method according to claim 1 wherein preparing the material includes differentiating the embryonic stem cells into chondroprogenitors.

6. The method according to claim 1 wherein causing the material to generate notochordal cells includes waiting for a predetermined period of time.

7. The method according to claim 1 wherein causing the material to generate notochordal cells includes causing the notochordal cells to differentiate into chondrocytes.

8. The method according to claim 1 further comprising using the notochordal cells as xenografts for restoration of degenerated discs.

9. The method according to claim 1 further comprising using differentiated chondrocyte cells as xenografts for restoration of degenerated discs.

10. The method according to claim 1 wherein the degenerated intervertebral disc being restored is a human disc.

11. A method for restoring a degenerated intervertebral human disc, said method comprising:
preparing a material including embryonic stem cells that includes pretreating the embryonic stem cells to encourage chondrogenic cell lineage and differentiating the embryonic stem cells into chondroprogenitors;
placing the material in the degenerated disc; and
causing the material to generate notochordal cells in the disc to regenerate the disc that includes causing the notochordal cells to differentiate into chondrocytes.

12. The method according to claim 11 wherein placing the material into the disc includes injecting the material into the disc using a syringe.

13. The method according to claim 11 wherein placing the material into the disc includes visualizing the material using lateral fluoroscopy.

14. The method according to claim 11 further comprising using the notochordal cells as xenografts for restoration of degenerated discs.

15. The method according to claim 11 further comprising using differentiated chondrocyte cells as xenografts for restoration of degenerated discs.

16. A method for creating embryonic stem cells, said method comprising:
preparing a material including embryonic stem cells that includes treating the embryonic stem cells to encourage a chondrogenic cell lineage and differentiating the embryonic stem cells into chondroprogenitors;
causing the material to generate notochordal cells; and
using the embryonic stem cell derived notochordal cells for research investigation and clinical applications.

17. The method according to claim 16 further comprising using the notochordal cells as xenografts for restoration of degenerated discs.

18. The method according to claim 16 further comprising using differentiated chondrocyte cells as xenografts for restoration of degenerated discs.
